(19) United States
(12) Patent Application Publication
(10) Pub. No.: US 2026/0158197 A1
(43) Pub. Date: Jun. 11, 2026

(54) OXYGENERATOR

(71) Applicant: CHARITÉ - UNIVERSITÄTSMEDIZIN BERLIN, Berlin (DE)

(72) Inventor: Leonid GOUBERGRITS, Berlin (DE)

(73) Assignee: CHARITÉ - UNIVERSITÄTSMEDIZIN BERLIN, Berlin (DE)

(21) Appl. No.: 18/707,547

(22) PCT Filed: Nov. 2, 2022
(86) PCT No.: PCT/EP2022/080548

§ 371 Date: May 3, 2024

(30) Foreign Application Priority Data
Nov. 3, 2021 (EP) 21206206.1

(51) Int. Cl. A61M 1/16; A61M 1/36; B01D 63/02; B01D 63/04

(52) U.S. Cl. CPC A61M 1/1698; A61M 1/3643; B01D 63/033; B01D 63/04; A61M 2207/00

(57) ABSTRACT

The invention relates to an oxygenator, comprising a plurality of hollow fibers (1) for receiving a flow (G, G1) of gas; and a blood compartment (2) formed adjacent the hollow fibers (1) for receiving a flow (B) of blood, the volume of the blood compartment (2) being associated with a blood priming volume (V) of the oxygenator (10). According to the invention, at least one element (3, 11) is arranged in the blood compartment (2) in such a way that it reduces the blood priming volume (V).

The invention relates to an oxygenator according to claim 1 and a method for fabricating an oxygenator according to claim 23.

An oxygenator is a device used to add oxygen to, and remove carbon dioxide from blood. It can be used in two principal modes: to imitate the function of the lungs in cardiopulmonary bypass (CPB), and to oxygenate blood in longer term life support (“extracorporeal membrane oxygenation”—ECMO). In a hollow fiber membrane oxygenator an oxygen containing gas flows through a bundle of hollow fibers, wherein sidewalls of the hollow fibers may comprise a thin gas permeable membrane separating the blood and gas flows (e.g. in the CPB circuit). Oxygen diffuses through the membrane into a flow of blood between the hollow fibers, while carbon dioxide diffuses through the membrane from the blood flow into the gas.

Currently, because of their low efficiency, the use of oxygenators ECMO may be associated with a set of complications including neurological injuries as subarachnoid hemorrhage, is-chemic infarctions or brain death. Bleeding occurs in 30 to 40% of patients receiving ECMO due to necessity in heparin infusion and platelet dysfunction. Further, known hollow fiber membrane oxygenators use fibers of about 300 μm diameter for oxygen flow, wherein the blood priming volume may be 40 to 50% of the total oxygenator volume. Such a blood priming volume is accompanied with a relatively high blood film thickness (e.g. of about 200 μm) and a relatively large required diffusion length LD (e.g. of 100 μm). Since the diffusion time is approxi-mately proportional to LD², the length of an oxygenator has to be large (e.g. at least about 100 mm) to achieve sufficient blood saturation.

The object of the invention is to allow for a more efficient and compact oxygenator.

According to the invention, an oxygenator is provided comprising
a plurality of hollow fibers for receiving a flow of gas; and
at least one blood compartment (e.g., a passage) formed adjacent the fibers for receiving a flow of blood, the volume of the blood compartment being associated with a blood priming volume of the oxygenator, wherein
at least one element arranged in the blood compartment in such a way that it reduces the blood priming volume.

Since the at least one element reduces the blood priming volume of the oxygenator (relative to the blood priming volume without the element), the blood film thickness (e.g. the average thickness of the blood flow) and thus the diffusion length required to obtain a satisfying (e.g. 100 %) blood saturation becomes smaller. Accordingly, the diffusion time, i.e. the time gas diffusing from the hollow fibers into the blood compartment needs to travel along the diffusion length, drops. Thus, the time the blood is required to flow through the blood compartment to achieve the desired saturation and subsequently the length of the blood compartment required for complete saturation (i.e. the saturation length) is reduced. This, in turn, may allow a more compact (miniaturized) design of the oxygenator; e.g., a shorter overall size (e.g., length) of the oxygenator. For example, the oxygenator according to the invention may be used to realize an implantable, efficient artificial lung device.

Moreover, the at least one element may be configured and arranged in such a way that a transverse flow component of the blood flow is increased (relative to a blood flow without the element). The “transverse flow component” in particular refers to a flow component perpendicular to a main flow component, wherein the main flow component may be parallel to a main extension direction of the hollow fibers. Thus, the transverse flow component in particular is directed towards the adjacent hollow fibers such that a convection flow towards the fibers is created. For example, due to the presence of the at least one element the magnitude of the transverse flow component may be at least of the same order of the magnitude of the main flow component or may be essentially the same. The increased transverse flow component may contribute in further reducing the diffusion time and thus may help to further reduce the size of the oxygenator.

The volume of the blood compartment forms at least a portion of the blood priming volume of the oxygenator, i.e. the volume of the oxygenator that is fillable with blood. It is conceivable that the volume of the blood compartment at least essentially is identical to the blood priming volume. However, the oxygenator may comprise other sections in addition to the blood compartment adjacent the hollow fibers that during operation of the oxygenator will fill with blood. It is possible that the blood compartment comprises or consists of a plurality of sub-volumes that are formed between neighboring fibers (e.g. between two neighboring fibers). The at least one element or a plurality of elements may be arranged in at least one of these subvolumes. Moreover, the oxygenator may comprise a housing in which the plurality of hollow fibers is arranged, wherein the blood compartment may comprise a first volume formed by the space between the hollow fibers and a second volume formed by a space between the hollow fibers and the housing.

The oxygenator, in particular, is a hollow fiber membrane oxygenator, i.e. sidewalls of the hollow fibers are formed by gas permeable membranes through which oxygen contained in the gas flow within the hollow fibers diffuses into the blood within the blood compartment and in the opposite direction carbon dioxide diffuses from the blood into the gas flow within the hollow fibers. The hollow fibers may form a fiber bundle arranged in a stacked or rolled-up configuration. For example, the hollow fibers form a fiber layer (e.g. a fiber mat) that is arranged in a rolled-up configuration.

The at least one element may be arranged movably relative to the hollow fibers. In particular, the at least one element may not be fixed relative to the hollow fibers or at the hollow fibers, but may be allowed to free-float in the blood flow. The at least one element that may be configured to rotate due to the gas flow and/or blood flow around it. This may prevent the existence of fixed areas with stagnating flow condition and thus may reduce the risk of thrombus formation.

The at least one element may be a hollow or a solid element and may have a closed outer surface. Further, the at least one element may comprise or consist of a plastic material, a glass or a ceramic material (or any other suited material). The material may be a biocompatible (in particular non-toxic) material. For example, the material complies with ISO 10993-18. Moreover, the material may be provided with a coating (e.g., a heparin or silicone coating).

According to an embodiment of the invention, the at least one element is a spherical element. In particular, the spherical element may not be attached to the hollow fibers and thus may be movable relative to the hollow fibers to float and rotate with the blood flow within the blood compartment, thereby reducing the risk of thrombus formation as set forth above. For example, a plurality of spherical elements may be arranged in the blood compartment, wherein the spherical elements at least essentially may be arranged in a close-packing configuration. It is conceivable that the number and the design of the spherical elements is chosen in such a way that they occupy at least half or at least two thirds of the original blood priming volume of the oxygenator.

The spherical elements at least essentially may have the same diameter. However, it is also conceivable that the spherical elements have different diameters. For example, the diameter of the spherical elements (or at least of some of the plurality of spherical elements) may be between 50 and 200 μm. An optimal diameter of the spherical elements may be derived from the requirement that a passage between the spherical elements should be formed whose diameter is not smaller than a predetermined minimum diameter. The predetermined minimum diameter may be set e.g. depending on the size of blood components such as the red blood cells. For example, if three of the spherical elements are in contact with one another, the spherical elements delimit a passage having a diameter D=2*R1*0.154, wherein R1 is the radius of the spherical elements. If D is chosen to be at least 10 μm it is derived that the diameter 2*R1 of the spherical element should be at least 65 μm.

The oxygenator according to the invention may further comprise an inlet port for supplying the flow of blood to the blood compartment and/or an outlet port permitting blood to exit the blood compartment; and a filter (e.g. a grating) associated with the inlet port for preventing the element to exit the blood compartment through the inlet port and/or a filter associated with the outlet port for preventing the element to exit the blood compartment through the outlet port.

According to another embodiment of the invention, the hollow fibers form a first fiber bundle, wherein the at least one element is a hollow fiber of a second fiber bundle, the fibers of the second fiber bundle being arranged at an angle relative to the fibers of the first fiber bundle. Thus, the hollow fiber of the second fiber bundle occupies at least a portion of the blood compartment volume, thereby reducing the blood priming volume. Of course, in addition elements movable relative to the first fiber bundle (such as the spherical elements mentioned above) may be arranged in the blood compartment.

The hollow fibers of the first fiber bundle may be in fluid communication with a first inlet port and the hollow fibers of the second fiber bundle may be in fluid communication with a second inlet port. Oxygen containing gas or pure oxygen may be supplied to the fibers of the first fiber bundle via the first inlet port, while oxygen containing gas or pure oxygen may be supplied to the fibers of the second fiber bundle via the second inlet port.

The fibers of the second fiber bundle may be arranged at an angle of at least 45° relative to the fibers of the first fiber bundle. For example, the fibers of the second fiber bundle are arranged at least essentially perpendicular to the fibers of the first fiber bundle.

Moreover, the first fiber bundle may form a first fiber layer and the second fiber bundle may form a second fiber layer, wherein the first and the second fiber layer are arranged in rolled-up (wound) configuration. For example, the fabrication of the oxygenator comprises arranging the first and the second fiber layer one above the other and rolling up the first and the second fiber layer. In particular, in the wound configuration and viewed in a radial direction, portions of the first fiber layer alternate with portions of the second fiber layer. The rolling up of the first and the second fiber layer may be carried out in such a way that endings of the first and the second hollow fibers remain accessible in the rolled-up state of the fiber layers in order to permit injection and ejection of gas into/from the hollow fibers. For example, rolling up the first and the second fiber layers is carried out by rotating the layers about a rotational axis that initially extends in a distance from the edges of the (spread out) fiber layers, e.g. through a middle (center) section of the layers. More particularly, the first and the second fiber layer may be rolled up from the center section towards the outer edges of the first and the second fiber layer. Further, the fibers of the first fiber bundle may not be woven with the fibers of the second fiber bundle.

The invention also relates to a method of fabricating an oxygenator, comprising:
providing a plurality of hollow fibers for receiving a flow of gas, wherein a blood compartment is formed adjacent the hollow fibers for receiving a flow of blood, the volume of the blood compartment being associated with a blood priming volume of the oxygenator;
arranging at least one element arranged in the blood compartment in such a way that it reduces the blood priming volume, wherein
the hollow fibers form a first fiber bundle and the at least one element is a hollow fiber of a second fiber bundle, the hollow fibers of the second fiber bundle being arranged at an angle relative to the fibers of the first fiber bundle.

Embodiments of the invention are described in more detail below with reference to the drawings, which show:

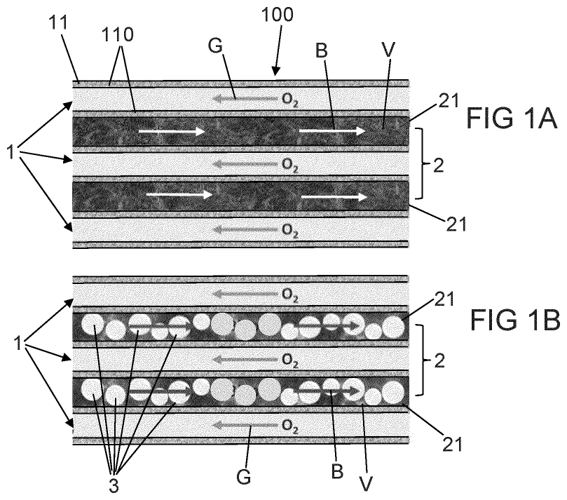

FIG. 1A schematically a cross-section of a portion of a conventional oxygenator;

FIG. 1B schematically a cross-section of a portion of an oxygenator according to a first embodiment of the invention;

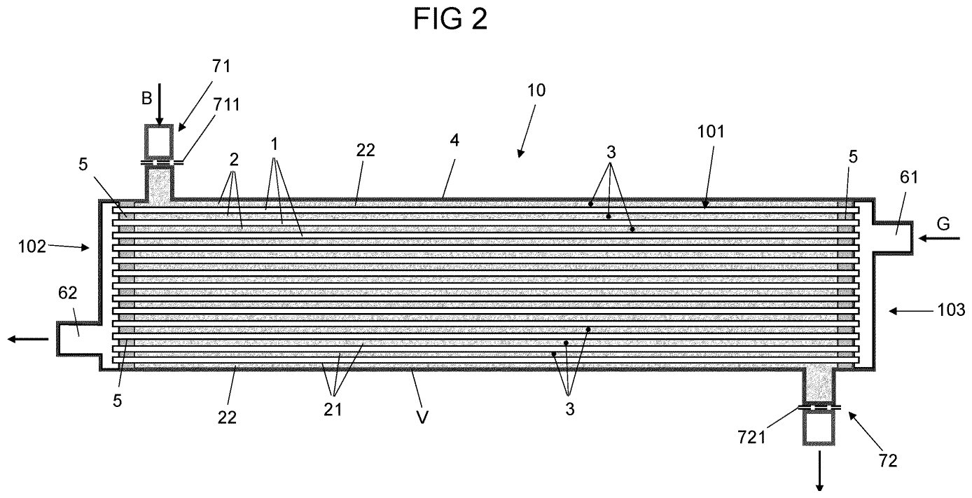

FIG. 2 a cross-section of an oxygenator according to a second embodiment of the invention;

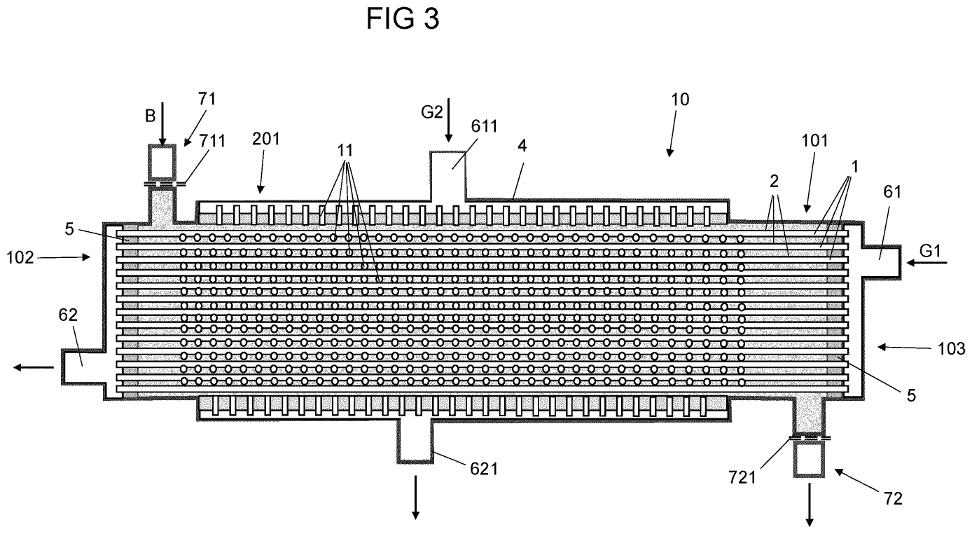

FIG. 3 a cross-section of an oxygenator according to a third embodiment of the invention;

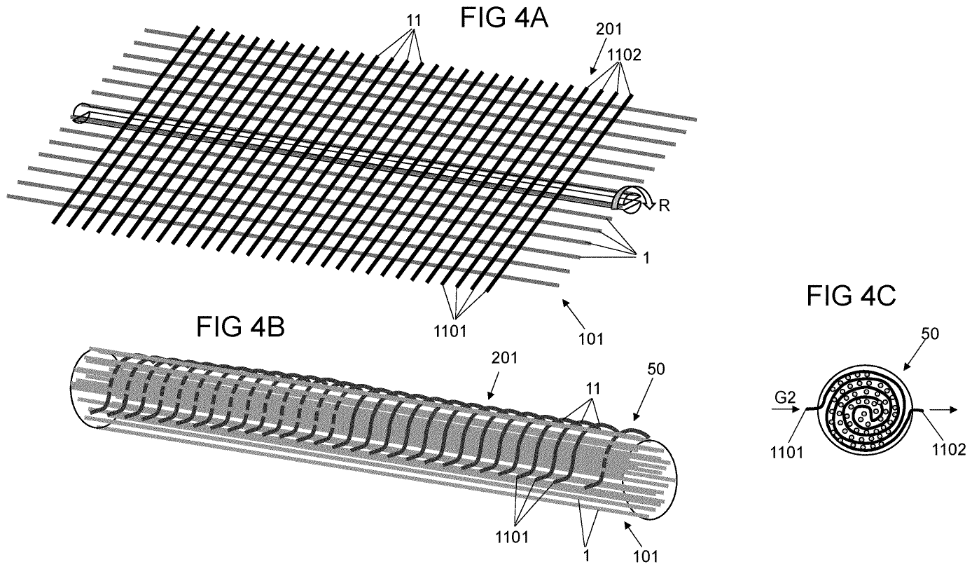

FIG. 4A fiber layers of the oxygenator shown in FIG. 3 in a spread out configuration;

FIG. 4B the fiber layers of FIG. 4A in a rolled-up configuration;

FIG. 4C a cross-section of the rolled-up configuration shown in FIG. 4B; and

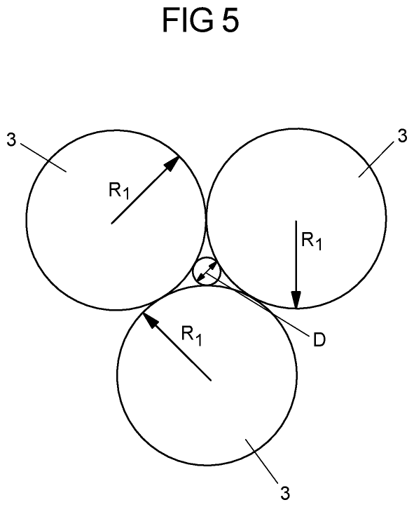

FIG. 5 spherical elements of an oxygenator according to an embodiment of the invention.

FIG. 1B shows a conventional type hollow fiber membrane oxygenator 100. The oxygenator 100 comprises a plurality of hollow fibers 1 for receiving a flow G of oxygen containing gas. Sidewalls 11 of the hollow fibers 1 consist of gas permeable membranes 110. Further, the oxygenator 100 comprises a blood compartment 2 formed between the hollow fibers 1 and configured for receiving a flow B of blood. The blood compartment 2 may comprise sub-blood compartments (sub-volumes) 21 located between 2 adjacent hollow fibers 1, the volume of blood compartment 2 corresponding to or forming a portion of the blood priming volume V of the oxygenator 100. As in principle known from the art, oxygen will diffuse from the gas flow conducted in the hollow fibers 1 into the blood stream within the blood compartment 2, while carbon dioxide diffuses from the blood compartment 2 into the hollow fibers 1.

The oxygenator 10 according to the invention and shown in FIG. 1B differs from the oxygenator depicted in FIG. 1A in that a plurality of spherical elements 3 are arranged within the blood compartment 2, i.e. within the sub-blood compartments 21 located between two neighboring hollow fibers 1. The spherical elements 3 reduce the fillable volume of blood compartment 2, i.e. because of the presence of the spherical elements 3 within the blood compartment 2, the oxygenator 10 shown in FIG. 1B has a smaller blood priming volume V than the oxygenator 100 of FIG. 1A. The smaller blood priming volume V reduces the average thickness of the blood stream (in particular the thickness of a blood film within the sub-blood compartments 21) such that the diffusion length required for a sufficient blood saturation is likewise reduced. This, in turn, allows a reduction of the overall length of the oxygenator as already set out above.

FIG. 2 shows an oxygenator 10 according to a second embodiment of the invention. The oxygenator 10 similarly to the one shown in FIG. 1B comprises a plurality of elongated straight hollow fibers 1 forming a fiber bundle 101. The hollow fibers 1 are arranged in a housing 4 in such a way that a blood compartment 2 is formed adjacent the hollow fibers 1. The blood compartment 2 defines at least a portion of the blood priming volume V of the oxygenator 10. In particular, the blood compartment 2 in that the blood priming volume V comprises sub-volumes 21 formed between neighboring hollow fibers 1 and may also comprise outer sub-volumes 22 present between outer hollow fibers 1 and housing 4. The blood priming volume V of the oxygenator 10 it is marked by a hatching in FIG. 2.

The fiber bundle 101 may be arranged in a rolled-up configuration to obtain a cylindrical shape, wherein housing 4 may have a corresponding cylindrical shape. Moreover, fiber bundle 101 maybe fixed to the housing 4 by an adhesive 5 applied at opposite endings 102, 103 of fiber bundle 101 (e.g. of the hollow fibers 1) and between adjacent hollow fibers 1. Other configurations of fiber bundle 101 are conceivable, e.g. a stacked configuration of the hollow fibers 1.

The oxygenator 10 further comprises a gas inlet port 61 being in fluid communication with the hollow fibers 1 and thus permitting oxygen containing gas G to enter the hollow fibers 1 (via the ending 103 of the fiber bundle 101). The gas inlet port 61 may be formed on a front surface of housing 4. Moreover, the oxygenator 10 comprises a gas outlet port 61 formed on an opposite end of the oxygenator 10, i.e. adjacent the ending 102 of fiber bundle 101. Gas can be released from the hollow fibers 1 via the outlet port 62. Blood enters the oxygenator 10, i.e. blood compartment 2, through a blood inlet port 71, and exits the blood compartment 2 via a blood outlet port 72. The ports 71 and 72 are arranged on opposite portions of a side wall of the housing 4. Other arrangements of ports 61, 62 and 71, 72, however, are of course possible.

Further, similar to FIG. 1B, blood compartment 2 is filled with a plurality of spherical elements 3 such that the blood priming volume V is reduced. The number of spherical elements 3 and the density of the spherical elements 3 (i.e. the number of spherical elements 3 per volume) may be chosen in such a way that a larger part of the original volume of blood compartment 2 is occupied by the spherical elements 3. For example, at least half of the original volume of blood compartment 2 is blocked by the spherical elements 3. The spherical elements 3 may be arranged in a close-packing configuration. For the sake of clarity, FIG. 2 only depicts some of the spherical elements 3.

Both the blood inlet port 71 and the blood outlet port 72 comprises a filter 711, 721 that pre-vents the spherical elements 3 from exiting the blood compartment 2 via the inlet port 71 and the outlet port 72, respectively.

FIG. 3 depicts an oxygenator 10 according to another embodiment of the invention. The oxygenator 10 of this embodiment comprises a first fiber bundle 101 comprising a plurality of first hollow fibers 1 arranged in a cylindrical housing 4. Similar to FIG. 2, a blood compartment 2 for receiving a flow of blood is formed adjacent the first hollow fibers 1. Further, a plurality of elements in the form of a plurality of second hollow fibers 11 is arranged in the blood compartment in such a way that the blood priming volume of the oxygenator 10 is reduced. The plurality of second hollow fibers 11 forms a second fiber bundle 201.

Both the first and the second fiber bundle 101, 201 are in a rolled-up configuration, wherein the first hollow fibers 1 at least essentially extend perpendicular to the second hollow fibers 11. The first hollow fibers 1 similar to FIG. 2 have an elongated straight shape and extend essentially parallel to a longitudinal axis of housing 4. Each one of the second hollow fibers 11 extends annularly and in a plane oriented perpendicular to the longitudinal axis of housing 4.

The oxygenator 10 identically to the embodiment of FIG. 2 comprises a blood inlet port 71 and a blood outlet port 72, the ports 71, 72 comprising filters 711, 721. Further, the oxygenator 10 comprises a first gas inlet port 61 for supplying a first flow G1 of oxygen containing gas to the first hollow fibers 1 of the first fiber bundle 101 and a second gas inlet port 611 for supplying a second flow G2 of oxygen containing gas to the second hollow fibers 11 of the second fiber bundle 201. The second gas inlet port 611 is separate from the first gas inlet port 61 and arranged in a distance from the first gas inlet port 61. Accordingly, the embodiment shown in FIG. 3 permits to supply gas independently to each one of the first and the second fiber bundle 101, 201. Gas will exit the first hollow fibers 1 via a first gas outlet port 62. Moreover, a second gas outlet port 621 is provided for releasing gas from the second hollow fibers 11 of the second fiber bundle 21.

The first gas inlet port 61 and the first gas outlet port 62 associated with the first hollow fibers 1 of the first fiber bundle 101 similarly to FIG. 2 are arranged on different sides of housing 4 located opposite to one another along the longitudinal axis of housing 4. The second gas inlet port 611 and the second gas outlet port 621 are arranged on different portions of the sidewall of housing 4 located opposite to one another along a direction perpendicular to the longitudinal axis of housing 4.

It should be noted that it is of course possible to arrange further elements within the blood compartment 2 in order to reduce the blood priming volume further. For example, in addition to the second hollow fibers 11, a plurality of (e.g. free-floating) elements such as the spherical elements 3 of FIG. 2 may be arranged within blood compartment 2.

FIGS. 4A to 4C depict different states of the first and the second fiber bundle 101, 201 of FIG. 3. More particularly, FIG. 4A shows the first and second fiber bundle 101, 201 in a flat, spread out configuration, while FIGS. 4B and 4C show the first and second fiber bundle 101, 201 in the wound-up configuration used to arrange the fiber bundles 101, 201 within the hollow cylindrical housing 4 of FIG. 3.

As shown in FIG. 4A, the first and the second fiber bundles 101, 201 initially are arranged as fiber layers disposed one above the other, wherein at least some of the first hollow fibers 1 of the first fiber bundle 101 may be connected to at least some of the second hollow fibers 11 of the second fiber bundle 21. In order to fabricate the rolled-up configuration shown in FIGS. 4B and 4C, the first and the second fiber bundle 101, 201 are rotated simultaneously around a rotational axis R that extends at least essentially through a middle portion of the first fiber bundle 101. The reason for using a rotational axis that extends in a distance from the outer hollow fibers of the first fiber bundle 101 is to allow first and second endings 1101, 1102 of the second hollow fibers 11 to remain accessible for gas input in the rolled-up configuration.

The rolled-up state created by rotating the first and the second fiber bundle 101, 201 around axis R is illustrated in FIG. 4B. In that state, the first and the second fiber bundle 101, 201 form a cylindrical body 50, wherein the fibers 1 of the first fiber bundle 101 extent elongated and parallel to the main (longitudinal) axis of the cylindrical body 50. The second hollow fibers 11 of the second fiber bundle 201 on the contrary extent at least partially in an annular manner around the main axis of the body 50. A cross-section of the body 50 of FIG. 4B (perpendicular to the main axis of the body 50) is shown in FIG. 4C. The first and second endings 1101, 1102 of the second hollow fibers 11 remain accessible such that the second gas flow G2 can be inserted via their first endings 1101 and ejected through their second endings 1102.

FIG. 5 shows a cross sectional view of three spherical elements 3 arranged in the blood compartment of an oxygenator between two adjacent hollow fibers (not shown), wherein the spherical elements 3 are in close contact with one another, i.e. any of the three spherical elements 3 touches the other two. A passage 31 is formed between the spherical elements 3, wherein blood will flow through the passage 31. Of course, the oxygenator may comprise several groups of three spherical elements 3 as shown in FIG. 5.

The diameter of the spherical elements 3 may be derived from a minimum diameter D associated with the passage 31 and required to allow a sufficient flow of blood through the passage 31. More particularly, D may be the diameter of a circular object that can be arranged between the spherical elements 3 so that it touches each one of them. As already set forth above, D may be predetermined based on the size of blood components such as the red blood cells. For example, if D is assumed to be at least 10 μm, the diameter 2*R1 of the spherical elements 3 should be at least 65 μm (R1 being the radius of the spherical elements 3).

1. Oxygenator, comprising
a plurality of hollow fibers (1) for receiving a flow (G, G1) of gas; and
a blood compartment (2) formed adjacent the hollow fibers (1) for receiving a flow (B) of blood, the volume of the blood compartment (2) being associated with a blood priming volume (V) of the oxygenator (10),
characterized by
at least one element (3, 11) arranged in the blood compartment (2) in such a way that it reduces the blood priming volume (V).

2. Oxygenator as claimed in claim 1, wherein sidewalls of the hollow fibers (1) are formed by a gas permeable membrane (110).

3. Oxygenator as claimed in claim 1 or 2, wherein the at least one element is configured and arranged in such a way that a transverse flow component of the blood flow is increased.

4. Oxygenator as claimed in any of the preceding claims, wherein the at least one element (3) is arranged movably relative to the hollow fibers (1).

5. Oxygenator as claimed in any of the preceding claims, wherein a plurality of elements is arranged in the blood compartment (2) to reduce the blood priming volume.

6. Oxygenator as claimed in any of the preceding claims, wherein the at least one element (3) is a spherical element.

7. Oxygenator as claimed in claim 6, wherein a plurality of spherical elements is arranged in the blood compartment (2), wherein the spherical elements at least essentially are arranged in a close-packing configuration.

8. Oxygenator as claimed in claim 6 or 7, wherein a plurality of spherical elements is arranged in the blood compartment (2), the spherical elements at least essentially having the same diameter.

9. Oxygenator as claimed in any of claims 6 to 8, wherein a plurality of spherical elements is arranged in the blood compartment (2), and wherein at least some of the spherical elements have different diameters.

10. Oxygenator as claimed in any of claims 7 to 9, wherein the diameter of the spherical element is between 50 and 200 μm.

11. Oxygenator as claimed in any of the preceding claims, further comprising
an inlet port (71) for supplying the flow (B) of blood to the blood compartment (2) and/or an outlet port (72) permitting blood to exit the blood compartment (2); and
a filter (711) associated with the inlet port (71) and preventing the element (3) to exit the blood compartment (3) through the inlet port (71) and/or a filter (721) associated with the outlet port (72) and preventing the element (3) to exit the blood compartment (2) through the outlet port (72).

12. Oxygenator as claimed in any of the preceding claims, wherein the hollow fibers (1) form a first fiber bundle (101), wherein the at least one element (11) is a hollow fiber of a second fiber bundle (201), the hollow fibers of the second fiber bundle (201) being arranged at an angle relative to the fibers (1) of the first fiber bundle (101).

13. Oxygenator as claimed in claim 12, wherein the hollow fibers (1) of the first fiber bundle (101) are in fluid communication with a first inlet port (61) and the hollow fibers of the second fiber (201) bundle are in fluid communication with a second inlet port (611).

14. Oxygenator as claimed in claim 12 or 13, wherein the angle is at least 45°.

15. Oxygenator as claimed in any of claims 12 to 14, wherein the first fiber bundle (101) forms a first fiber layer and the second fiber bundle (201) forms a second fiber layer,

16. Oxygenator as claimed in claim 15, wherein the first and the second fiber layers are arranged in wound configuration.

17. Oxygenator as claimed in claim 16, wherein the wound configuration is generated by arranging the first and the second fiber layer one above the other and rolling up the first and the second fiber layer.

18. Oxygenator as claimed in claim 17, wherein in the wound configuration and viewed in a radial direction, portions of the first fiber layer alternate with portions of the second fiber layer.

19. Oxygenator as claimed in any of claims 16 to 18, wherein the wound configuration is generated by rolling up the first and the second fiber layer about a rotational axis (R) that extends in a distance from outer edges of the fiber layers.

20. Oxygenator as claimed in claim 19, wherein the rotational axis (R) extends through a center section of the first and the second fiber layer such that the first and the second fiber layer are rolled up from the center section towards the outer edges of the first and the second fiber layer.

21. Oxygenator as claimed in any of claims 16 to 20, wherein in the wound configuration endings (1101, 1102) of the first and the second hollow fibers are accessible to permit injection and ejection of gas into/from the hollow fibers.

22. Oxygenator as claimed in any of claims 12 to 21, wherein the fibers of the first fiber bundle (101) are not woven with the fibers of the second fiber bundle (201).

23. Method for fabricating an oxygenator as claimed in any of the preceding claims, comprising:
providing a plurality of hollow fibers (1) for receiving a flow (G, G1) of gas, wherein a blood compartment (2) is formed adjacent the hollow fibers (1) for receiving a flow (B) of blood, the volume of the blood compartment (2) being associated with a blood priming volume (V) of the oxygenator (10);
arranging at least one element (3, 11) arranged in the blood compartment (2) in such a way that it reduces the blood priming volume (V), wherein
the hollow fibers (1) form a first fiber bundle (101) and the at least one element (11) is a hollow fiber of a second fiber bundle (201), the hollow fibers of the second fiber bundle (201) being arranged at an angle relative to the fibers (1) of the first fiber bundle (101).
